(19) United States
(12) Patent Application Publication
(10) Pub. No.: US 2022/0039698 A1
(43) Pub. Date: Feb. 10, 2022

(54) Method and System for Enhancing Monitoring Glucose

(71) Applicants: HOSPITAL CLÍNIC DE BARCELONA, Barcelona (ES); INSTITUT D'INVESTIGACIONS BIOMEDIQUES AUGUST PI I SUNYER (IDIBAPS), Barcelona (ES); UNIVERSITAT POLITÈCNICA DE VALÈNCIA (UPV), Valencia (ES); CENTRO DE INVESTIGACIÓN BIOMÉDICA EN RED (CIBER), Madrid (ES); UNIVERSITAT DE GIRONA, Girona (ES)

(72) Inventors: Alejandro José LAGUNA SANZ, Madrid (ES); Jorge BONDÍA COMPANY, Valencia (ES); José Luis DIEZ RUANO, Valencia (ES); Josep VEHÍ CASELLAS, Girona (ES); Marga GIMÉNEZ, Barcelona (ES); Ignacio CONGET, Barcelona (ES)

(21) Appl. No.: 16/985,749

(22) Filed: Aug. 5, 2020

(51) Int. Cl. A61B 5/145; A61B 5/1495; A61B 5/01; A61B 5/00; A61M 5/142; A61M 5/172

(52) U.S. Cl. CPC A61B 5/14532; A61B 5/1495; A61B 5/01; A61B 5/681; A61B 2560/0223; A61M 5/142; A61M 5/1723; A61B 5/7221; A61B 5/4866

(57) ABSTRACT

Methods and systems for enhancing glucose monitoring are disclosed that allow for more precise control of the glucose level by taking into account new control parameters. The disclosed methods include obtaining an initial CGM value (CGM) from a CGM sensor, obtaining a set of parameters from a wearable device, calculating an error (E) in the initial CGM value (CGM) using a regression algorithm based upon the initial CGM value and the obtained set of parameters, and calculating an enhanced CGM value (eCGM) according to the formula eCGM=CGM−E. The error (E) is calculated according to the formula: E=θ*p, wherein E is the calculated error, θ represents the data obtained from the wearable device after removing a baseline value of each wearable, and p represents regression parameters based upon an error value obtained in a training population.

### FIELD OF THE INVENTION

The present disclosure relates to the technological field of glucose control. Specifically, the disclosure is directed to a more precise control of glucose concentration values in humans.

An object of the present disclosure is to provide methods for enhancing monitoring glucose, which allows for more precise control of the glucose level by taking into account new control parameters.

A second object of the present disclosure is to provide a system for enhancing monitoring glucose configured to carry out the disclosed methods.

### BACKGROUND OF THE INVENTION

Diabetes Mellitus is a disease characterized by a dysregulation of the natural homeostasis of glucose concentration in the body. It is projected to have a prevalence of 9.9% in the global population by 2045 (629 million people affected), and the estimated yearly cost in healthcare expenditure by the same year is 776 billion USD. Approximately 10% of the people who have diabetes suffer from type 1 diabetes, which has a very early onset, usually manifesting during childhood or adolescence, and requires intensive supervision and treatment of the disease.

Type 1 Diabetes (T1D) is defined by the inability of the beta cells of the pancreas to produce insulin in the bloodstream partially or totally, which leads to abnormally high levels of blood glucose. This problem worsens during periods of meal ingestion, in which glucose is poured into the bloodstream through the gut, or during physical activity or stress, which alter uptake or production of glucose in different parts of the body.

An Artificial Pancreas (AP) is a device that feeds continuous glucose measurements into an insulin pump in real time and continuously adjusts insulin dosage delivered to the patient. It is one of the most promising solutions to the complications of T1D, and several prototypes have been under development over the last few years, including commercially-available devices.

Physical activity has been linked to changes in glucose trends and variability in patients with diabetes. The accuracy of glucose measurement is of great importance for the correct managing of diabetes, since it drives the decision making of the algorithms behind it.

The accuracy of Continuous Glucose Monitoring (CGM) devices has been observed to be affected during exercise periods. Some studies have shown that, for example, Dexcom G4 and Medtronic Enlite devices' accuracy dropped during aerobic physical activity when compared to reference glucose measurements. Increased CGM error has been found consistently during aerobic exercise, even among the more recently distributed devices in the market, such as the artificial pancreas Medtronic 670G (provided with Guardian™ Sensor 3), and the Flash Glucose Monitoring device Abbott Freestyle Libre.

A study showed that the accuracy of Medtronic Enlite 2 devices dropped both during aerobic and anaerobic exercise, but only the results during aerobic exercise were significantly different. This conclusion has also been reported in the literature before, which suggests an underlying problem with the mechanism of glucose estimation in the subcutaneous tissue during periods of physical activity.

Exercise monitoring is widespread today in most parts of the world, with many different devices designed and marketed to provide estimation of the intensity, type, and duration of physical activity.

The use of wearable devices to improve T1D management has been reviewed before, and many studies have been conducted by adding different isolated wearable variables into AP controller algorithms, supervision algorithms, or prediction and classification algorithms.

The influence of wearable signals has already been evaluated in the context of their possible implementation in an artificial pancreas system, showing that the amount of information carried by each signal changes depending on the type of exercise performed and placing special importance on the estimation of total energy spent by the physical activity.

### SUMMARY

The present disclosure is related to methods for enhancing monitoring glucose which allow an improvement of the accuracy of the estimation of glucose in a subject. The disclosed methods advantageously compensate the estimation error shown during exercise periods.

For doing that, exercise monitoring devices are used, so as to provide information to a control module, which is configured for performing a real-time correction of the shift in glucose estimation during physical activity.

The disclosed methods are based on a regression model, which improves the accuracy of a CGM signal, obtained previously by a CGM sensor, during the exercise period, and, at the same time, maintaining the baseline accuracy during the resting period.

The methods for enhancing monitoring glucose of the invention include a step of obtaining an initial glucose or CGM value by using a CGM sensor, which will be considered the baseline value. The CGM sensor uses algorithmic rules to fit the chemical measurements of the subcutaneous probe into relatively accurate plasma glucose values.

Then, a set of parameters is obtained from a wearable device. Preferably, said parameters could comprise at least one of a metabolic equivalent or a skin temperature, which has proven to be the most significant in enhancing the accuracy of the CGM estimation.

Having obtained CGM data and wearable parameters, an error (E) value is obtained with respect to the initial CGM value (CGM). The error (E) value is calculated by using a regression algorithm, according to the formula:

E=θ*p Eq. 1

In Eq. 1, E is the calculated error, θ represents the data obtained from the wearable device, after removing a baseline value of each wearable, and p represent regression parameters based upon an error value obtained in a training population.

Preferably, the regression model is calibrated so as to obtain the regression parameters, by using data obtained for a training population, wherein the plasma glucose is known, and, thus, the error value could be inferred. Then, the regression parameters are calculated from Eq. 1, being known the error (E) value and the wearable parameters.

Then, an enhanced CGM value (eCGM) is calculated according to the Eq. 2:

eCGM=CGM−E Eq. 2

The method of the invention could comprise also a step of calculating an amount of insulin needed according to the calculated enhanced CGM value (eCGM).

Systems for enhancing glucose monitoring are also disclosed. The disclosed systems include at least one CGM sensor, for obtaining CGM data, at least one wearable device, for obtaining wearable parameters, and a calculation unit, connected to the at least one CGM sensor and the at least one wearable device.

The calculation unit of the system is configured to perform a set of instructions, which include obtaining an initial CGM value (CGM) from the CGM sensor, obtaining a set of wearable parameters, from the wearable device, which comprises at least one of a metabolic equivalent or a skin temperature, from the wearable device.

The calculation unit is further configured to calculate an error (E) value with respect to the initial CGM value (CGM), by using a regression algorithm, according to the Eq. 1:

E=θ*p Eq. 1

wherein E is the calculated error, θ represents the data obtained from the wearable device and p represent regression parameters based upon an error value obtained in a training population.

Then, the calculation unit is also configured to calculate an enhanced CGM value (eCGM), according to the Eq. 2:

eCGM=CGM−E Eq. 2

Optionally, the disclosed systems can also include a glucose controller connected to the calculation unit and an insulin pump, yielding to an artificial pancreas system. Then, the calculation unit would be further adapted to calculate an amount of insulin needed according to the calculated enhanced CGM value (eCGM). Then, the calculation unit sends instructions to the insulin pump for dispensing insulin according to the amount of insulin needed calculated by the calculation unit.

As explained, the integration of physiological signals in CGM sensors for monitoring plasma glucose improves the accuracy of the measured signal, especially during exercise periods.

The presently disclosed regression model (composed of only two parameters) achieves a reduction in the MARD during aerobic exercise that practically nullified the influence of the exercise on the CGM estimation error.

### BRIEF DESCRIPTION OF THE DRAWINGS

To complement the description being made and in order to aid towards a better understanding of the characteristics of the present claims, in accordance with a preferred example of practical embodiment thereof, a set of drawings is attached as an integral part of said description wherein, with illustrative and non-limiting character, the following has been represented:

FIG. 1—shows a graph with the correlations between wearable parameters.

FIG. 2—shows a graph with the variation in MARD values on removing wearable parameters from a list of regression parameters contained in vector p sequentially, for the exercise period (top) and global (bottom).

FIG. 3A—shows a graph with the Clarke error grid analysis for an exercise period with the initial CGM value.

FIG. 3B—shows a graph with the Clarke error grid analysis for an exercise period with the enhanced CGM value.

FIG. 3C—shows a graph with the Clarke error grid analysis for a resting period with the initial CGM value.

FIG. 3D—shows a graph with the Clarke error grid analysis for a resting period with the enhanced CGM value.

FIG. 4A—shows a graph with the Parkes error grid analysis for an exercise period with the initial CGM value.

FIG. 4B—shows a graph with the Parkes error grid analysis for an exercise period with the enhanced CGM value.

FIG. 4C—shows a graph with the Parkes error grid analysis for a resting period with the initial CGM value.

FIG. 4D—shows a graph with the Parkes error grid analysis for a resting period with the enhanced CGM value.

FIG. 5A—shows a graph with the ISO acceptable region for an exercise period with the initial CGM value.

FIG. 5B—shows a graph with the ISO acceptable region for an exercise period with the enhanced CGM value.

FIG. 5C—shows a graph with the ISO acceptable region for a resting period with the initial CGM value.

FIG. 5D—shows a graph with the ISO acceptable region for a resting period with the enhanced CGM value.

### DETAILED DESCRIPTION

The method of the invention allows to monitor in a precise way the evolution of the glucose in a subject. For that, uses a regression model for estimating an error value in a measure given by a CGM sensor. Therefore, an enhanced CGM value is calculated.

As previously disclosed, CGM sensors are not quite precise when measuring glucose in a subject during physical activity. Therefore, the error value is calculated using data obtained from a wearable device comprising at least one sensor.

A wearable device is intended for measuring parameters related to physical activity. In an exemplary embodiment the method of the invention uses wearable devices such as:
Fitbit Charge HR™ (Fitbit, San Francisco, Calif., USA): a physical activity monitor that tracks several exercise signals: (1) heart rate, (2) steps, (3) floor level, (4) Metabolic equivalents (METs), and (5) calories burned.
Microsoft® Band 2 (Microsoft, Redmond, Wash., USA): a multipurpose device that tracks physiological variables such as: (1) heart rate, (2) steps, (3) galvanic skin response, (4) skin temperature and (5) movements.
Polar heart rate monitor, Model RCX3® (Polar, Kempele, Finland): a commercially-established heart rate monitor.

A physical activity could be characterized by measuring using wearable devices one or more of the following parameters:
HR: Heart Rate; measured in beats per minute; can be used to estimate exercise intensity.
ST: STeps; the number of steps walked.
LV: LeVel; the number of floors of stairs climbed.
ME: MEts; the metabolic equivalent of tasks; an estimation of exercise intensity normalized for each patient.
CA: CAlories; calories burned.
TM: TeMperature; skin temperature.
GS: Galvanic Skin Response; an estimation of the skin electrodermal activity, also known as the conductance level of the skin.
MO: MOvements; accumulated movement magnitude registered by the accelerometer.

Then, after have being collected data from the wearable devices, an error value is estimated. For that, a regression algorithm is applied to the data obtained from the wearable devices.

In particular, a linear regression algorithm can be used. Thus, a vector of regression parameters is defined, which correlates the obtained data from the wearable devices and the estimated error. The linear regression algorithm can be determined by the Eq. 1:

E=θ*p Eq. 1

In Eq. 1, E is the calculated error, θ represents the data obtained from the wearable device, after removing a baseline value of each wearable, and p represent regression parameters based upon an error value obtained in a training population. The intercept of the model was forced to be zero, in this case, so as to better compensate only the error observed in the exercise period.

An enhanced CGM value is then calculated by subtracting the estimated error value from a measure of an initial CGM value obtained by a CGM sensor, as represented by the Eq. 2. The enhanced CGM value represents a more precise estimation of glucose during periods of aerobic physical activity.

eCGM=CGM−E Eq. 2

In a preferred embodiment of the invention, only two parameters are taken into account from the data obtained from the wearable devices: ME and TM.

These parameters have been demonstrated to improve the preciseness of the glucose monitoring.

In an alternative embodiment, just the ME parameter could be used for calculating the enhanced CGM value. Being, therefore, the calculation process easier and faster, and not reducing in an important way the preciseness of the glucose monitoring.

### EXAMPLES

### Example 1

In a first example, a dataset was obtained for analyzing the performance of an exercise-challenged closed-loop controller in T1D people. A total of six participants were enrolled at the Clinic University Hospital of Barcelona. The protocol was approved by the Ethics Committee of the hospital. Criteria for eligibility were: (1) age between 18 and 60 years old, (2) Body Mass Index (BMI) between 18 and 30 kg/m2, (3) Glycated Hemoglobin A1c (HbA1C) between 6.0% and 8.5%, and (4) use of Continuous Subcutaneous Insulin Infusion (CSII) for at least six months. Exclusion criteria included: (1) pregnancy, (2) use of experimental drug or devices in the past 30 days, (3) onset of progressive fatal diseases, (4) hypoglycemia unawareness, (5) drug or alcohol abuse, and (6) other systematic diseases other than T1D, including hepatic, neurological, and endocrine-related illnesses. A summary of the patient demographics is shown in Table 1.

TABLE 1

| | |
|---|---|
| Number of Patients (Females) | 6 (1) |
| Age (years) | 36.7 ± 8.9 |
| HbA1c (%) | 7.9 ± 0.5 |
| BMI (kg/m2) | 24.6 ± 1.0 |
| Time with T1D (years) | 25.2 ± 12.7 |
| Time with CSII (years) | 4.8 ± 1.7 |

Each participant underwent three aerobic tests. The subjects used Paradigm Veo® insulin pumps, and two Enlite-2® (Medtronic Minimed, Northridge, Calif., USA) CGM sensors were inserted in different parts of the abdomen the day before the trial by the subjects. Plasma glucose (PG) as a reference value was measured using the YSI 2300 Stat Plus Glucose Analyzer (YSI Incorporated Life Sciences, Yellow Springs, Ohio, USA), with a frequency of 15 min. A total of 27 sets of data were available by the end of the study. The exercise schedules were as follows:

Aerobic routine: Patients exercised doing three bouts of 15 min on a cycloergometer at 60% of the patients maximum capacity with five minutes of rest between them.

The exercise intensity was converted into units of heart rate, which was different for each patient and calculated as follows:

$$HR ( e x ) \_i = \frac{Int}{100} \cdot ( HR ( max ) \_i - HR ( 0 ) \_i ) + HR ( 0 ) \_i \qquad Eq . 3$$

In Eq. 3, HR(ex)_i is the intensity of the exercise in terms of heart rate for patient i, Int stands for the percentage intensity of the exercise (60 for aerobic exercise), and HR(max)_i stands for the maximum heart rate of patient i, defined as [226−(age_i)] for women, and [220−(age_i)] for men, where age_i is the age of patient i in years. Lastly, HR(0)_i is the resting heart rate of patient i as measured at the beginning of the experiment.

By using the training population, a training set was obtained, wherein the glucose value is known for each subject. Then, an error value is obtained according to the formula:

E_i(k)=CGM_i(k)−PG_i(k) Eq. 4

In Eq. 4, E_i (k) represents a CGM estimation error for data stream i at timestamp k. CGM_i (k) represents the CGM data obtained by a CGM sensor and PG_i (k) represents plasma glucose data measured with a YSI 2300 Stat Plus Glucose Analyzer (YSI Incorporated Life Sciences, Yellow Springs, Ohio, USA). Each data stream i is a time series corresponding to a particular experiment and CGM signal.

CGM accuracy was evaluated using the Mean Absolute Relative Deviation (MARD), calculated as:

$$MARD\_i = \frac{{\sum }_{k = 1}^{n} \frac{\vert E\_i ( k ) \vert}{PG\_i ( k )}}{n} \qquad Eq . 5$$

In Eq. 5, MARD_i is the average percent error of the data stream i and n is the total number of samples available for i.

Then, data is obtained from wearable devices. The data obtained were pre-processed by removing the baseline value of each wearable signal and trial from the signal itself. The baseline of each signal was calculated as the median value of that particular variable for each data stream evaluated only in the resting period. It has to be noted that, in an out-of-clinic environment, this value is trivial to obtain since it would be the equilibrium value of the signal during resting or sleeping periods.

Wearable devices could obtain, in this example, the following parameters, wherein the first letter of the abbreviation used is the initial letter of the wearable device that provided that signal, and the second and third letters are an abbreviation of the name of the signal:
FHR: Fitbit Heart Rate; measured in beats per minute; can be used to estimate exercise intensity.
FST: Fitbit STeps; the number of steps walked.
FLV: Fitbit LeVel; the number of floors of stairs climbed.
FME: Fitbit MEts; the metabolic equivalent of tasks; an estimation of exercise intensity normalized for each patient.
FCA: Fitbit CAlories; calories burned.
MHR: Microsoft Band Heart Rate.
MTM: Microsoft Band TeMperature; skin temperature.
MGS: Microsoft Band Galvanic Skin Response; an estimation of the skin electrodermal activity, also known as the conductance level of the skin.
MST: Microsoft Band STeps; number of steps walked.
MMO: Microsoft Band MOvements; accumulated movement magnitude registered by the accelerometer.
PHR: Polar Heart Rate.

The availability of the parameters obtained from the wearable devices was calculated, as the complementary value of the percentage of missing samples, varied from signal to signal. Table 2 shows the availability of each parameter obtained from the wearable devices.

TABLE 2

| | FHR | FST | FLV | FME | FCA | MHR | MTM | MGS | MST | MMO | PHR |
|---|---|---|---|---|---|---|---|---|---|---|---|
| A (%) | 96.4 | 100 | 100 | 100 | 100 | 78.7 | 73.3 | 78.7 | 14.7 | 73.3 | 55.3 |

The data obtained from the wearable devices, were evaluated so as to reduce the order of the regression algorithm. The parameters analyzed were obtained from the three wearable devices disclosed previously, i.e. Fitbit Charge HR™, Microsoft® Band 2, Polar heart rate monitor, Model RCX3®. Therefore the obtained parameters were: FHR, FST, FLV, FME, FCA, MHR, MTM, MGS, MST, MMO and PHR.

In order to determine which parameters of the wearable devices were more relevant, said parameters were selected based on the following criteria:
Lack of availability of synchronous data to CGM-YSI pairs obtained previously.
Lack of representation of the type of physical activity performed in the study: cycloergometer aerobic exercise.
High correlation between wearable signals. A high correlation between two signals implies that the amount of information carried by each one of those signals is similar; thus, one of them can be eliminated without hindering the predictive capabilities of the model.
MARD improvement evaluated in the validation sets. A wearable parameter was removed from the input set if the MARD for the validation sets (both in the exercise period and in global terms) was improved by removing that wearable parameter alone.

If a variable complied with the above rules, it was removed from the inputs of the multiple regression model, and the model was fit again to the data and the outputs re-evaluated, until no improvement of the error was achieved by removing any of the inputs.

Then, the predictive power of the model was validated by performing a non-exhaustive leave nine-out cross-validation of the model. Out of the set of 27 streams of data, two thirds (i.e., 18 randomly-chosen streams out of 27) were selected as a fitting set, and the remaining data were used to validate the fitted model.

Model accuracy was evaluated on the validation set, the results stored, and the process was repeated, randomly selecting new fitting and validation sets. This process was repeated 20 times. All the accuracy metrics shown onward were those averaging the results throughout all the 20 validation sets of nine streams of data.

In the selection of data, first, availability was considered. Therefore, any parameter having 30% or more samples not usable, such as missing samples, disconnections or out of bounds samples, was discarded from the model. This elimination of parameters leaded to discard MST and PHR, due to their low availability.

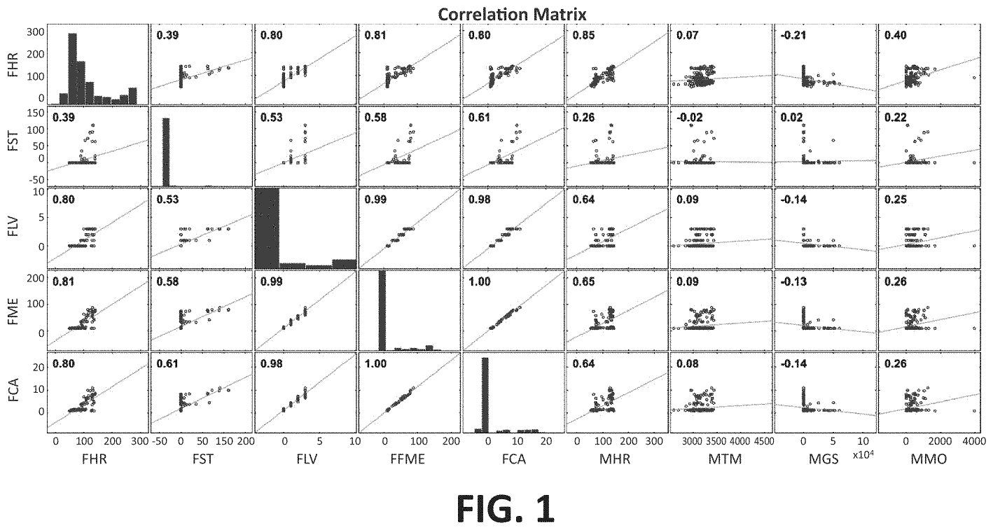

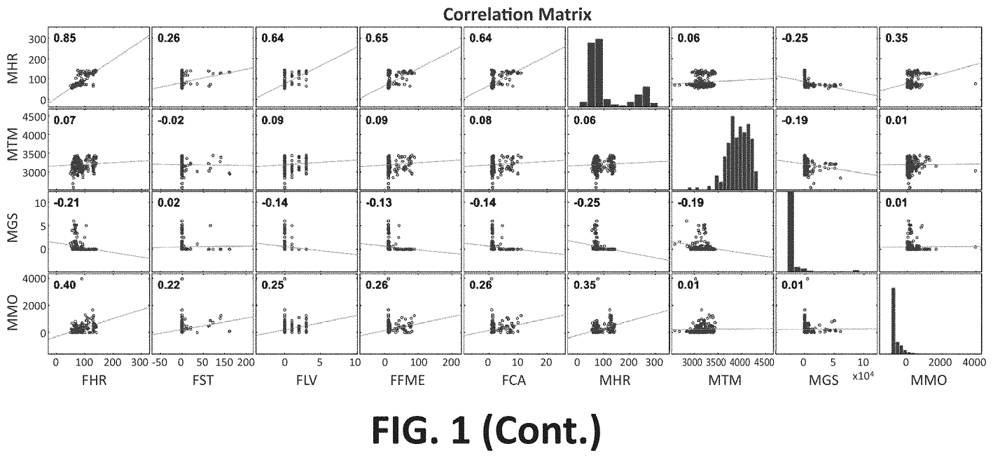

Then, the correlation between parameters was evaluated, as shown in FIG. 1. If two or more parameters show a high correlation, then they contain almost the same information, and removing one will not have a great impact in the accuracy of the calculation of glucose. Thus, the correlations between parameters derived from the accelerometer sensor, i.e. FLV, FME, and FCA, showed that those three variables carried essentially the same information.

In this case, FME was considered to be a more relevant variable due to its continuous nature, while FLV is discrete, and due to the fact that it was normalized by using the baseline energy consumption of each patient. Therefore, a linear model using this parameter as an input would require less individualization than one using the other parameters. Thus, it was decided to discard FLV and FCA from the model fit.

Similarly, FHR and MHR presented a high correlation, wherein the R value was 0.85. This is coherent with the nature of those variables, since both of them estimated the same physiological value, i.e. the heart rate.

In this case, it was decided that FHR was more relevant than MHR for the linear model due to its higher availability, as shown in Table 2. Thus, MHR was discarded as an input to the model.

Finally, FST was deemed irrelevant for the type of exercise performed in the trials. FST estimates the intensity of physical activity and quantifies that value in the number of steps walked by the patient. The physical activity from this study (cycloergometer) rendered the patient static for most of the exercising period, which would result in inaccurate estimations of the steps. Furthermore, the histogram of the signal FST in FIG. 1 shows that most of the FST values laid very close to zero. For these reasons, FST was removed from the input parameter array.

After removing FST from the inputs list, the input parameter array comprised the following parameters: FHR, FME, MTM, MGS and MMO.

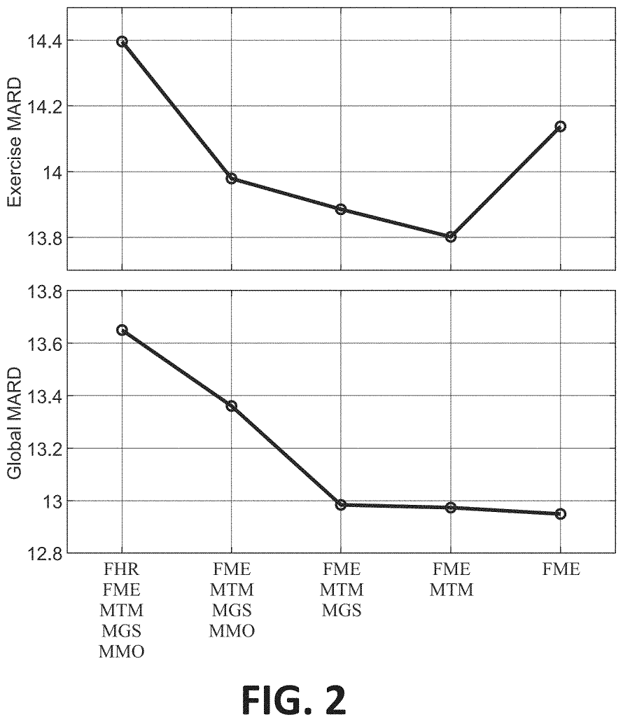

Using those parameters as inputs, the linear model was fit, and the results of the MARD improvement in the validation sets were recorded. A single input was removed one at a time, the model's performance recorded, and the cycle repeated, removing another input and using the others. The input elimination that further worsened the MARD in the exercise period, or the one that most worsened the MARD for the whole trial period in case of equal exercise MARD values, was permanently eliminated. The sequence of eliminations and the results on the MARD reduction are shown in FIG. 2.

By removing the FHR and MMO parameters, the global MARD, and also the MARD for the exercise period, were reduced, thus, improving the model. Then, by removing MGS, the global MARD reduction was minimal, but a reduction is achieved in the MARD for the exercise period. These differences are small when compared to the MARD reduction achieved by utilizing any of those models versus not using any model to compensate for exercise (17.46%→13.8% and 17.46%→14.14%). Thus, a model with only one parameter could be used. In this particular case, the model is simpler, especially when looking at the fact that both input signals in this example came from different devices on the market: FME was provided by the Fitbit device, and MTM was extracted from the Microsoft device.

The last two signals, FME and MTM, were remained. By removing MTM, a minimal global MARD reduction was achieved, while the MARD in the exercise period increases. For that reason, MTM was not removed from the input parameter array.

Having these two parameters as inputs, the model was fit resulting in the coefficients shown in Table 3. Table 3 shows a value of the coefficient p, as the average value in the case explained wherein a cross-validation process was carried out, and in a case wherein all available data, not separating any for validations, is used for fitting the model.

TABLE 3

| Parameter | pFME | pMTM |
|---|---|---|
| Average parameter in | 0.33 | −0.0017 |
| cross-validation | | |
| Parameter fitting all data | 0.297 | −0.0053 |

MARD results for the proposed model are shown in Table 4. Also, a Coefficient of Variation (CV) is shown to evaluate glycemic variability, being CV defined as:

$$C V = 100 \cdot \frac{S D ( C G M )}{mean ( CGM )} \qquad Eq . 6$$

In Eq. 6, SD represents the standard deviation and mean the mean value.

TABLE 4

| | | Original | Enhanced | |
|---|---|---|---|---|
| | | CGM | CGM | p-Value |
| MARD | Exercise period | 17.46% | 13.8% | <0.05 |
| | Resting Period | 12.75% | 12.88% | 0.75 |
| | Total | 13.61% | 12.97% | 0.4 |
| | CV | 17% | 16% | 0.17 |

As shown in Table 4, MARD during the exercise period was reduced drastically and significantly, from 17.46% to 13.8%, which was much closer to the average MARD of the original CGM of 13.61%. The total MARD (CGM error in the whole dataset, not separating resting and exercise periods) was reduced slightly, from 13.61% to 12.97%, and the error during the resting period was increased marginally, from 12.75% to 12.88%, not resulting in a significant difference than in the original CGM.

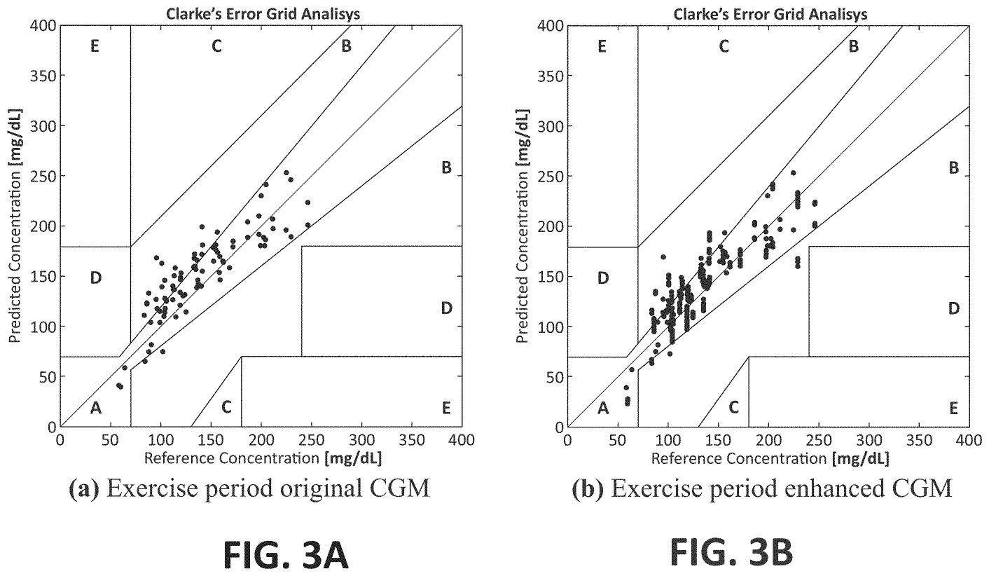

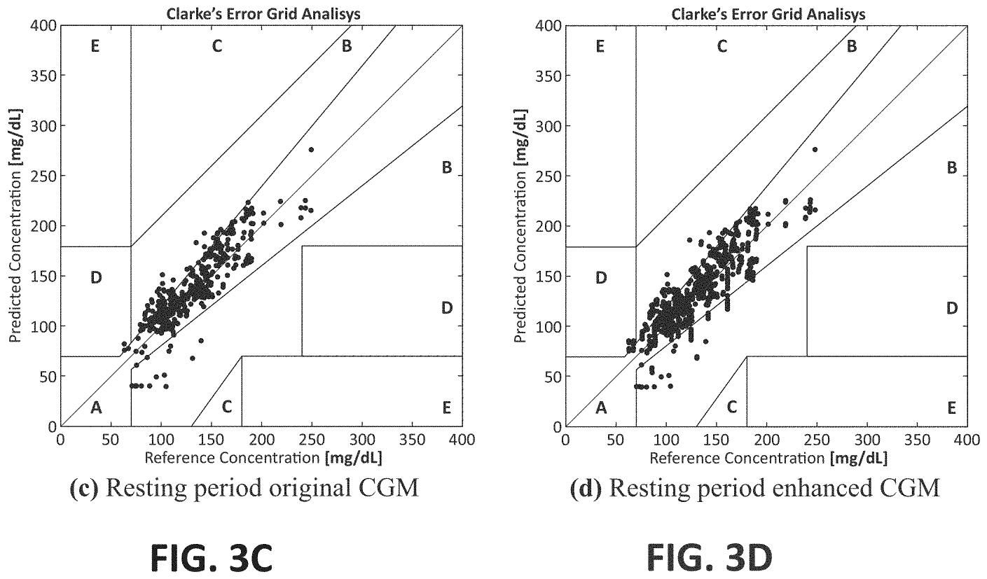

The validation data were evaluated using different acceptability criteria. FIGS. 3A-3D show a Clarke error grid analysis (EGA) of said data. Since 20 iterations of the fit-validate cycle were performed, there were several data points corresponding to the same plasma glucose value, displaying vertical patterns in the eCGM subplots. This also means that the point density of the eCGM subplots was much higher than those of the original CGM. The results showed a displacement towards the A zone, especially during exercise, as a response to the proposed correction. The original CGM already had 100% of the samples in the A+B zone during exercise, and without hindering that performance, eCGM achieved a shift of the data points towards the A zone for the exercise period (72.4%→85.7%). Therefore, an improvement is achieved in the accuracy of the model. Table 5 shows the numeric values of the results for the Clarke error grid analysis.

TABLE 5

| | Zone | | A | B | C | D | E |
|---|---|---|---|---|---|---|---|
| CGM-PG | Exercise | CGM | 72.4 | 27.6 | 0 | 0 | 0 |
| pair | Period | eCGM | 85.7 | 14.3 | 0 | 0 | 0 |
| allocation | Resting | CGM | 85.6 | 14.2 | 0 | 0.2 | 0 |
| (%) | Period | eCGM | 84.3 | 15.1 | 0 | 0.6 | 0 |
| | Total | CGM | 82.6 | 17.2 | 0 | 0.2 | 0 |
| | | eCGM | 84.6 | 15 | 0 | 0.4 | 0 |

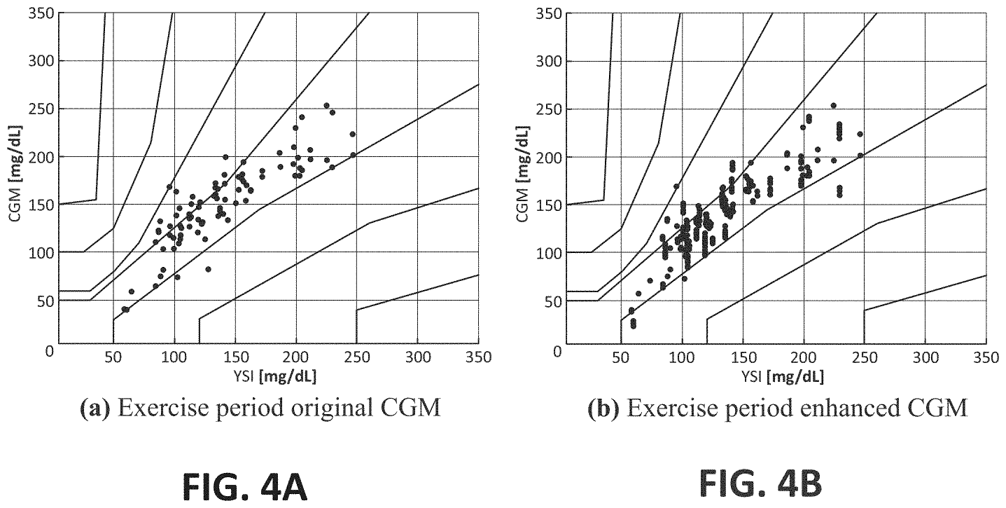

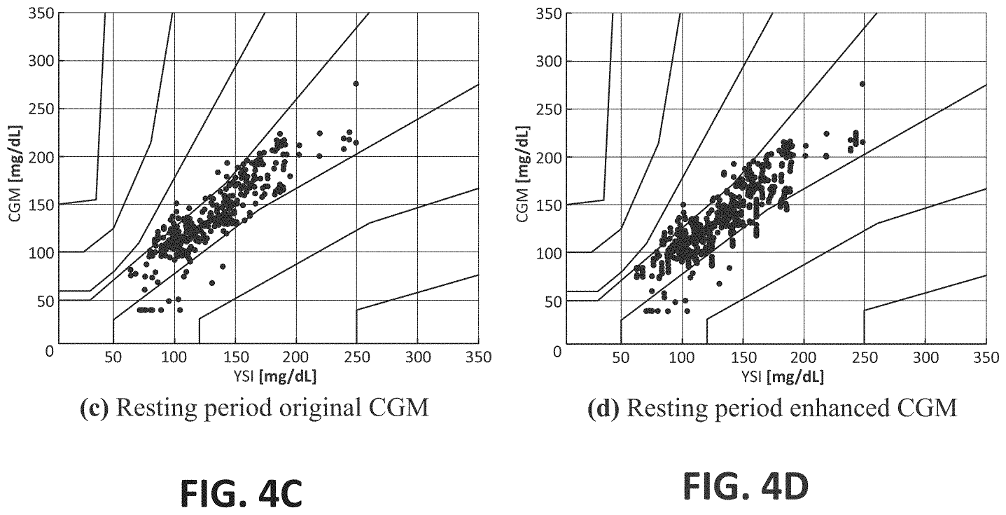

In addition to the Clarke EGA analysis, FIGS. 4A-4D show the results of the Parkes EGA, which classifies the same pairs of data into different zones, with the advantage of showing more continuous transitions between adjacent zones in the PG-CGM plane. Table 6 shows the numeric results for the Parkes EGA analysis, wherein an increase of points in Zone A (79.2%→89.2%) is observed, while there are not any point in Zones C, D and E.

TABLE 6

| | Zone | | A | B | C | D | E |
|---|---|---|---|---|---|---|---|
| CGM-PG | Exercise | CGM | 79.2 | 20.8 | 0 | 0 | 0 |
| pair | Period | eCGM | 89.2 | 10.8 | 0 | 0 | 0 |
| allocation | Resting | CGM | 90.5 | 9.5 | 0 | 0 | 0 |
| (%) | Period | eCGM | 87.7 | 12.3 | 0 | 0 | 0 |
| | Total | CGM | 88 | 12 | 0 | 0 | 0 |
| | | eCGM | 88 | 12 | 0 | 0 | 0 |

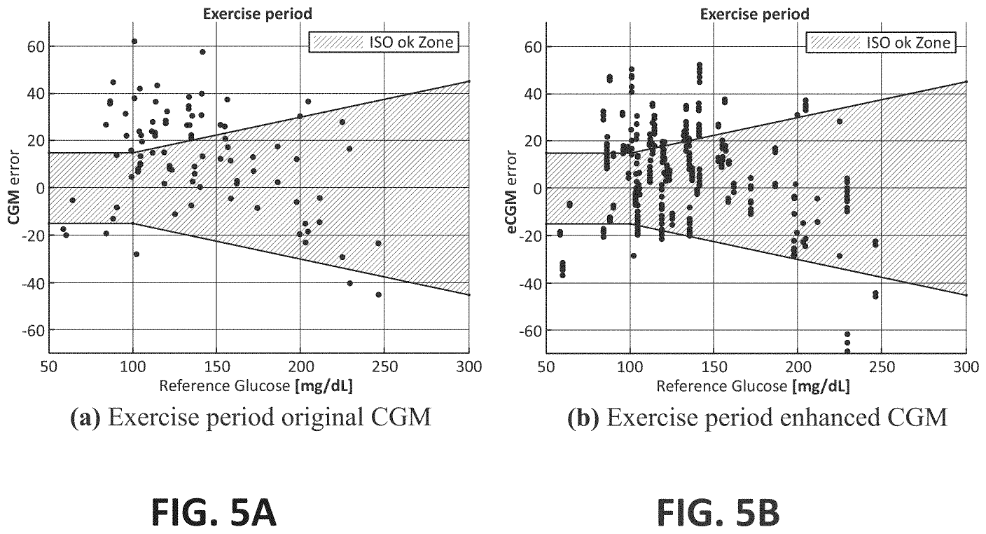

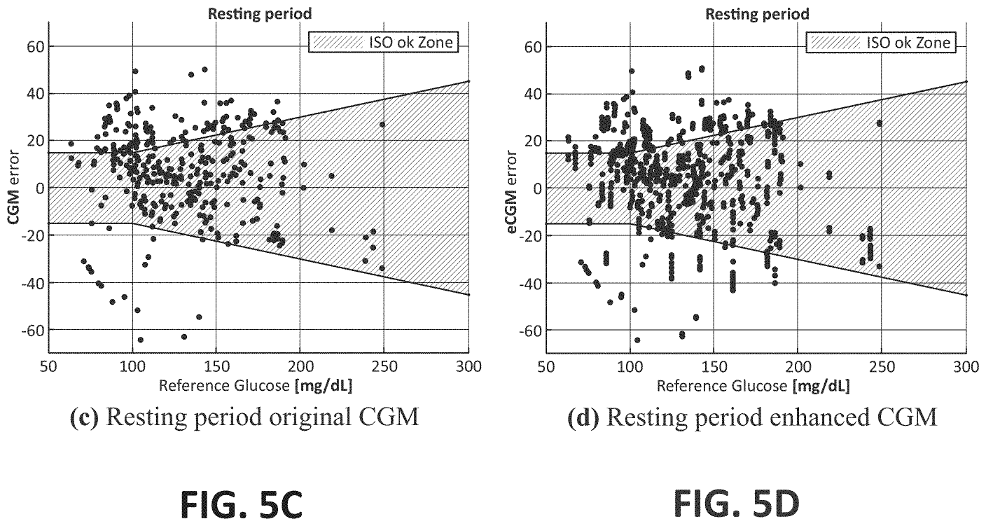

In FIGS. 5A-5D, the results for an ISO standard are shown. The ISO classification had only two zones, separating the CGM-PG pairs into “OK” estimations and “not OK” estimations. Table 7 shows the total number of samples in each category. Therefore, ISO results also showed better results for eCGM than the initial CGM signal, increasing the amount of “OK” samples 55.2%→64.4% during the exercise period, as well as increasing the total samples in the ISO zone 66.6%→68.8%.

TABLE 7

ISO Valid Samples

| | Original CGM | Enhanced CGM |
|---|---|---|
| Exercise Period | 55.23% | 64.4% |
| Resting Period | 69.9% | 70.1% |
| Total | 66.6% | 68.8% |

What is claimed is:

1. A method for enhancing monitoring glucose, the method comprising:
obtaining an initial CGM value (CGM) from a CGM sensor;
obtaining a set of parameters, from a wearable device;
calculating an error (E) in the initial CGM value (CGM), by using a regression algorithm based upon the initial CGM value and the obtained set of parameters, according to the formula:
E=θ*p,
wherein E is the calculated error, θ represents the data obtained from the wearable device, after removing a baseline value of each wearable, and p represent regression parameters based upon an error value obtained in a training population; and
calculating an enhanced CGM value (eCGM), according to the formula:
eCGM=CGM−E.

2. The method of claim 1, wherein the regression parameters (p) are obtained by means of a calibration process in which data obtained for a training population is used.

3. The method of claim 1, wherein the wearable parameters are at least one of metabolic equivalent or a skin temperature.

4. The method of claim 1, further comprising a step of calculating an amount of insulin needed according to the calculated enhanced CGM value (eCGM).

5. A system for enhancing monitoring glucose, the system comprising:
at least one CGM sensor, for obtaining a CGM value;
at least one wearable device, for obtaining a set of wearable parameters; and
a calculation unit connected to the at least one CGM sensor and the at least one wearable, and adapted to perform the following instructions:
obtaining an initial CGM value (CGM) from the CGM sensor;
obtaining a set of parameters from the wearable;
calculating an error (E) in the initial CGM value (CGM), by using a regression algorithm, according to the formula:
E=θ*p,
wherein E is the calculated error, θ represents the data obtained from the wearable and p is a vector of regression parameters; and
calculating an enhanced CGM value (eCGM), according to the formula:
eCGM=CGM−E.

6. The system of claim 5, wherein the at least one wearable device is configured to obtain a metabolic equivalent or a skin temperature.

7. The system of claim 5, further comprising an insulin pump connected to the calculation unit, which is further adapted to calculate an amount of insulin needed according to the calculated enhanced CGM value (eCGM), and wherein the insulin pump dispenses insulin according to the amount of insulin needed calculated by the calculation unit.
